(19) United States
(12) Patent Application Publication
(10) Pub. No.: US 2026/0263237 A1
(43) Pub. Date: Sep. 10, 2026

(54) Acetabular Component And Extraction Tool For Use Therewith

(71) Applicant: Mayo Foundation for Medical Education and Research, Rochester, MN (US)

(72) Inventors: Rafael J. Sierra, Rochester, MN (US); Katherine E. Mallett, Rochester, MN (US)

(21) Appl. No.: 19/556,647

(22) Filed: Mar. 4, 2026

Related U.S. Application Data
(60) Provisional application No. 63/768,522, filed on Mar. 7, 2025.

(51) Int. Cl. A61F 2/46; A61B 17/17; A61F 2/30; A61F 2/34; A61F 2/36

(52) U.S. Cl. CPC A61F 2/4609; A61B 17/1746; A61F 2/30724; A61F 2/34; A61F 2/3662; A61F 2002/4619; A61F 2002/4629

(57) ABSTRACT

A hip joint prosthetic system comprises an acetabular component and an extraction tool for the acetabular component. The acetabular component includes: an acetabular cup including a wall terminating in a rim, the wall and the rim defining a cavity of the acetabular cup, the wall having an outer surface and an inner surface within the cavity, the acetabular cup having a first pair of spaced apart first projections extending outwardly from the outer surface of the wall and a second pair of spaced apart second projections extending outwardly from the outer surface of the wall, the first pair of spaced apart first projections defining a first channel that extends to the rim, and the second pair of spaced apart second projections defining a second channel that extends to the rim; a first spacer removably positioned in the first channel; and a second spacer removably positioned in the second channel.

### CROSS-REFERENCES TO RELATED APPLICATIONS

This application is based on, claims benefit of, and claims priority to U.S. Application No. 63/768,522, filed on Mar. 7, 2025, which is hereby incorporated by reference herein in its entirety for all purposes.

### STATEMENT REGARDING FEDERALLY SPONSORED RESEARCH

Not Applicable.

### BACKGROUND OF THE INVENTION

### 1. Field of the Invention

The invention relates to a bone joint prosthetic system, and more particularly to a hip joint prosthetic system having an acetabular component and an extraction tool for use with the acetabular component.

### 2. Description of the Related Art

Total hip arthroplasty (THA) is a well-established procedure for restoring hip function in patients with degenerative joint diseases, fractures, and other orthopedic conditions. Dual mobility implants are currently being used in 10% of primary and 30% of revision THA. Recently, there has been an increase in the use of cemented dual mobility components in the United States, especially at the time of revision THA when cementation of dual mobility is preferred into a well fixed shell, into a highly porous acetabular cup, or antiprotrusio ring. The difficulty lies when extraction of dual mobility acetabular cups is needed, for example when femoral head dislocation occurs and the dual mobility needs revision or with infection.

What is needed therefore is a dual mobility acetabular cup that allows for ease of removal by an extraction tool.

### SUMMARY OF THE INVENTION

The present disclosure meets the foregoing needs by providing a hip joint prosthetic system having an acetabular component and an extraction tool for use with the acetabular component. An acetabular cup allows for partial cementation, providing the same mechanical strength as full cementation, but also allows for cementless areas that ease removal by use of an extraction tool.

In one aspect, the present disclosure provides a prosthetic system for providing motion between a pelvis and a femur of a hip joint of a subject. The system comprises: an acetabular cup including a wall terminating in a rim, the wall and the rim defining a cavity of the acetabular cup, the wall having an outer surface and an inner surface within the cavity of the acetabular cup, the acetabular cup having a first pair of spaced apart first projections extending outwardly from the outer surface of the wall and a second pair of spaced apart second projections extending outwardly from the outer surface of the wall, the first pair of spaced apart first projections defining a first channel that extends to the rim, and the second pair of spaced apart second projections defining a second channel that extends to the rim; a first spacer removably positioned in the first channel; and a second spacer removably positioned in the second channel.

In another aspect, the present disclosure provides an extraction tool for removing an existing acetabular component from the acetabulum, wherein the acetabular component includes an acetabular cup including a wall terminating in a rim, the wall and the rim defining a cavity of the acetabular cup, the wall having an outer surface and an inner surface within the cavity of the acetabular cup. The extraction tool comprises: a shaft; a first arm; a second arm, wherein the first arm and the second arm are attached to the shaft in a spaced apart relationship such that the first arm can engage the outer surface of the wall of the acetabular cup and the second arm can engage the outer surface of the wall of the acetabular cup; and an adjustable retention mechanism engaging a first section of the first arm and a second section of the second arm, the retention mechanism opposing opening forces exerted on the first arm and the second arm.

In yet another aspect, the present disclosure provides an extraction tool for removing an existing acetabular component from the acetabulum, wherein the acetabular component includes an acetabular cup including a wall terminating in a rim, the wall and the rim defining a cavity of the acetabular cup, the wall having an outer surface and an inner surface within the cavity of the acetabular cup. The extraction tool comprises: a frame; a first block moveable with respect to the frame; and a second block moveable with respect to the frame, wherein the first block and the second block are located in a spaced apart relationship in an interior space of the frame such that the first block can engage the outer surface of the wall of the acetabular cup and the second block can engage the outer surface of the wall of the acetabular cup.

In still another aspect, the present disclosure provides an extraction tool for removing an existing acetabular component from the acetabulum, wherein the acetabular component includes an acetabular cup including a wall terminating in a rim, the wall and the rim defining a cavity of the acetabular cup, the wall having an outer surface and an inner surface within the cavity of the acetabular cup, the acetabular cup having at least two throughholes in the wall of the acetabular cup. The extraction tool comprises: a frame; a cap secured to the frame, wherein the cap has a wall with at least two holes, the cap being dimensioned such that a distal surface of the cap can engage the inner surface within the cavity of the acetabular cup; a pin in each hole of the cap; and a shaft dimensioned to move each of the pins into an associated throughhole of the acetabular cup when the distal surface of the cap engages the inner surface within the cavity of the acetabular cup.

In another aspect, the present disclosure provides a kit for preparing a prosthetic system for providing motion between a pelvis and a femur of a hip joint of a subject. The kit comprises: an acetabular cup including a wall terminating in a rim, the wall and the rim defining a cavity of the acetabular cup, the wall having an outer surface and an inner surface within the cavity of the acetabular cup, the acetabular cup having a first pair of spaced apart first projections extending outwardly from the outer surface of the wall and a second pair of spaced apart second projections extending outwardly from the outer surface of the wall, the first pair of spaced apart first projections defining a first channel that extends to the rim, and the second pair of spaced apart second projections defining a second channel that extends to the rim; a first spacer removably positioned in the first channel; a second spacer removably positioned in the second channel; and an extraction tool comprising a shaft, a first arm, and a second arm, the first arm and the second arm being attached to the shaft in a spaced apart relationship such that the first arm can engage the outer surface of the wall of the acetabular cup between the first pair of spaced apart first projections and the second arm can engage the outer surface of the wall of the acetabular cup between the second pair of spaced apart second projections.

In still another aspect, the present disclosure provides a kit for preparing a prosthetic system for providing motion between a pelvis and a femur of a hip joint of a subject. The kit comprises: an acetabular cup including a wall terminating in a rim, the wall and the rim defining a cavity of the acetabular cup, the wall having an outer surface and an inner surface within the cavity of the acetabular cup, the acetabular cup having a first pair of spaced apart first projections extending outwardly from the outer surface of the wall and a second pair of spaced apart second projections extending outwardly from the outer surface of the wall, the first pair of spaced apart first projections defining a first channel that extends to the rim, and the second pair of spaced apart second projections defining a second channel that extends to the rim; a first spacer removably positioned in the first channel; a second spacer removably positioned in the second channel; and an extraction tool comprising a frame, a first block moveable with respect to the frame, a second block moveable with respect to the frame, wherein the first block and the second block are located in a spaced apart relationship in an interior space of the frame such that the first block can engage the outer surface of the wall of the acetabular cup between the first pair of spaced apart first projections and the second block can engage the outer surface of the wall of the acetabular cup between the second pair of spaced apart second projections.

In yet another aspect, the present disclosure provides a kit for preparing a prosthetic system for providing motion between a pelvis and a femur of a hip joint of a subject. The kit comprises: an acetabular cup including a wall terminating in a rim, the wall and the rim defining a cavity of the acetabular cup, the wall having an outer surface and an inner surface within the cavity of the acetabular cup; a drill guide dimensioned such that a distal surface of the drill guide can engage the inner surface within the cavity of the acetabular cup, the drill guide having at least two passageways for directing a boring instrument into the wall of the acetabular cup for creating at least two throughholes in the wall of the acetabular cup; and an extraction tool comprising: (i) a frame, (ii) a cap secured to the frame, wherein the cap has a wall with at least two holes, the cap being dimensioned such that a distal surface of the cap can engage the inner surface within the cavity of the acetabular cup, (iii) a pin in each hole of the cap, and (iv) a shaft dimensioned to move each of the pins into an associated throughhole of the acetabular cup when the distal surface of the cap engages the inner surface within the cavity of the acetabular cup.

In yet another aspect, the present disclosure provides a method for replacing at least a part of a hip joint of a subject. The method comprises: (a) providing an acetabular component including: (i) an acetabular cup including a wall terminating in a rim, the wall and the rim defining a cavity of the acetabular cup, the wall having an outer surface and an inner surface within the cavity of the acetabular cup, the acetabular cup having a first pair of spaced apart first projections extending outwardly from the outer surface of the wall and a second pair of spaced apart second projections extending outwardly from the outer surface of the wall, the first pair of spaced apart first projections defining a first channel that extends to the rim, and the second pair of spaced apart second projections defining a second channel that extends to the rim, (ii) a first spacer removably positioned in the first channel, and (iii) a second spacer removably positioned in the second channel; (b) preparing a pelvis of the hip joint for receiving the acetabular component; (c) applying bone cement to the outer surface of the wall of the acetabular cup; (d) implanting the acetabular component in the pelvis; (e) removing the first spacer from the first channel; and (f) removing the second spacer from the second channel.

In still another aspect, the present disclosure provides a method of revising a total hip arthroplasty. The method comprises: (a) providing an extraction tool comprising a shaft, a first arm, and a second arm, the first arm and the second arm being attached to the shaft in a spaced apart relationship; (b) positioning the first arm and the second arm of the extraction tool in contact with an existing acetabular component implanted in an acetabulum, wherein the acetabular component includes: (i) an acetabular cup including a wall terminating in a rim, the wall and the rim defining a cavity of the acetabular cup, the wall having an outer surface and an inner surface within the cavity of the acetabular cup, the acetabular cup having a first pair of spaced apart first projections extending outwardly from the outer surface of the wall and a second pair of spaced apart second projections extending outwardly from the outer surface of the wall, the first pair of spaced apart first projections defining a first channel that extends to the rim, and the second pair of spaced apart second projections defining a second channel that extends to the rim; and (c) engaging the first arm of the extraction tool with the outer surface of the wall of the acetabular cup between the first pair of spaced apart first projections; (d) engaging the second arm of the extraction tool with the outer surface of the wall of the acetabular cup between the second pair of spaced apart second projections; and (e) applying a force to the extraction tool such that the acetabular component is removed from the acetabulum.

In yet another aspect, the present disclosure provides a method of revising a total hip arthroplasty, wherein the method comprises: (a) providing an extraction tool comprising a frame, a first block moveable with respect to the frame, and a second block moveable with respect to the frame, the first block and the second block being located in a spaced apart relationship in an interior space of the frame; (b) positioning the first block and the second block of the extraction tool in contact with an existing acetabular component implanted in an acetabulum, wherein the acetabular component includes: (i) an acetabular cup including a wall terminating in a rim, the wall and the rim defining a cavity of the acetabular cup, the wall having an outer surface and an inner surface within the cavity of the acetabular cup, the acetabular cup having a first pair of spaced apart first projections extending outwardly from the outer surface of the wall and a second pair of spaced apart second projections extending outwardly from the outer surface of the wall, the first pair of spaced apart first projections defining a first channel that extends to the rim, and the second pair of spaced apart second projections defining a second channel that extends to the rim; (c) engaging the first block of the extraction tool with the outer surface of the wall of the acetabular cup between the first pair of spaced apart first projections; (d) engaging the second block of the extraction tool with the outer surface of the wall of the acetabular cup between the second pair of spaced apart second projections; and (e) applying a force to the extraction tool such that the existing acetabular component is removed from the acetabulum.

In still another aspect, the present disclosure provides a method of revising a total hip arthroplasty, wherein the method comprises: (a) providing an extraction tool comprising: (i) a frame, (ii) a cap secured to the frame, the cap having a wall with at least two holes, (iii) a pin in each hole of the cap, and (iv) a shaft dimensioned to move each of the pins outward in its associated hole of the cap; (b) positioning a distal surface of the cap in contact with an existing acetabular component implanted in an acetabulum, wherein the acetabular component includes: (i) an acetabular cup including a wall terminating in a rim, the wall and the rim defining a cavity of the acetabular cup, the wall having an outer surface and an inner surface within the cavity of the acetabular cup, the acetabular cup having at least two throughholes in the wall of the acetabular cup; (c) translating the shaft of the extraction tool to move each of the pins into an associated throughhole of the acetabular cup; and (d) applying a force to the extraction tool such that the existing acetabular component is removed from the acetabulum.

In an example embodiment of the present disclosure, a dual mobility hip cup assembly is comprised of a small ceramic head, large polyethene head, outer metal liner, and a titanium implant. This assembly is designed to prevent dislocations and improve patient mobility. With total hip revisional arthroplasty cases, surgeons have struggled to remove the cemented dual mobility metal liner. Current practices to remove the metal liner are strenuous and invasive, leaving metal particulate matter in the body which can lead to surgical complications. To address current practices, surgeons see an urgent need for innovation surrounding instrumentation for extraction. The present disclosure provides an orthopedic extraction instrument to remove the implanted dual mobility metal liner cup from the implanted acetabular implant assembly. Current practices are extremely time-consuming, potentially harmful, and invasive, leaving metallic particulate inside the body which can lead to potential complications or require removal of the implanted acetabular component without removal of the metal liner and severe bone destruction. The present disclosure provides innovative ideas to address means for improving the current practice.

These and other features, aspects, and advantages of the present invention will become better understood upon consideration of the following detailed description, drawings, and appended claims.

### BRIEF DESCRIPTION OF THE DRAWINGS

FIG. 1 is a back view of one non-limiting embodiment of an acetabular component of a prosthetic system of the present disclosure.

FIG. 2 is a side view of the acetabular component of FIG. 1.

FIG. 3 is a front view of the acetabular component of FIG. 1.

FIG. 4 is a partial front view of the acetabular component of FIG. 1.

FIG. 5 is another side view of the acetabular component of FIG. 1.

FIG. 6 is a side view of one non-limiting embodiment of an extraction tool of the present disclosure engaging the acetabular component of FIG. 1.

FIG. 7 is a side view of another non-limiting embodiment of an extraction tool of the present disclosure engaging the acetabular component of FIG. 1.

FIG. 8 is a side view of yet another non-limiting embodiment of an extraction tool of the present disclosure engaging the acetabular component of FIG. 1.

FIG. 9 is a perspective view of one non-limiting embodiment of a femoral component for use in a prosthetic system of the present disclosure.

FIG. 10 is a perspective view of another non-limiting embodiment of acetabular components and a femoral component for use in a prosthetic system of the present disclosure.

FIG. 11 is a perspective view of still another non-limiting embodiment of an extraction tool of the present disclosure suitable for engaging the acetabular component of FIG. 1.

FIG. 12 is a cross-sectional view of the extraction tool of FIG. 11 taken along line 12-12 of FIG. 11.

FIG. 13A is a side view of the extraction tool of FIG. 11 in a first position before insertion over the acetabular component of FIG. 1.

FIG. 13B is a side view of the extraction tool of FIG. 11 in a second position tightening over the acetabular component of FIG. 1.

FIG. 13C is a side view of the extraction tool of FIG. 11 in a third position for removing the acetabular component of FIG. 1.

FIG. 14 is a perspective view of yet another non-limiting embodiment of an extraction tool of the present disclosure suitable for engaging the acetabular component of FIG. 1.

FIG. 15A is a perspective view of a drill guide of the extraction tool of FIG. 14 before engaging the acetabular component.

FIG. 15B is a cross-sectional view of the drill guide of the extraction tool of FIG. 15A before engaging the acetabular component of FIG. 15A taken along line 15B-15B of FIG. 15A.

FIG. 16A is a perspective view of the drill guide of the extraction tool of FIG. 15A after insertion in the acetabular component.

FIG. 16B is a cross-sectional view of the drill guide of the extraction tool of FIG. 15A after insertion in the acetabular component of FIG. 15A taken along line 16B-16B of FIG. 16A.

FIG. 16C is a back view of the acetabular component of FIG. 1 with the spacers removed and mounting holes having been prepared in the wall of the acetabular component.

FIG. 16D is a partial front view of the acetabular component of FIG. 1 with the spacers removed and mounting holes having been prepared in the wall of the acetabular component.

FIG. 17A is a front view of the extraction tool of FIG. 14 after insertion in the acetabular component of FIG. 15A.

FIG. 17B is a side view of the extraction tool of FIG. 14 after insertion in the acetabular component of FIG. 15A.

FIG. 17C is a cross-sectional view of the extraction tool of FIG. 14 after insertion in the acetabular component of FIG. 15A taken along line 17B-17B of FIG. 17A.

FIG. 18A is a front view of the extraction tool of FIG. 14 extending pins into holes in the acetabular component of FIG. 15A.

FIG. 18B is a side view of the extraction tool of FIG. 14 extending pins into holes in the acetabular component of FIG. 15A.

FIG. 18C is a cross-sectional view of the extraction tool of FIG. 14 extending pins in the acetabular component of FIG. 15A taken along line 18B-18B of FIG. 18A.

FIG. 19A is a front view of the extraction tool of FIG. 14 when removing the acetabular component of FIG. 15A.

FIG. 19B is a side view of the extraction tool of FIG. 14 when removing the acetabular component of FIG. 15A.

FIG. 19C is a cross-sectional view of the extraction tool of FIG. 14 when removing the acetabular component of FIG. 15A taken along line 19B-19B of FIG. 19A.

FIG. 20A is an exploded perspective view of a test fixture and test base of a testing device for measuring mechanical properties when removing the acetabular component of FIG. 15A.

FIG. 20B is a perspective view of the test fixture and the acetabular component of FIG. 20A.

FIG. 21 is a perspective view of a lever arm of the testing device of FIG. 20A engaging the acetabular component.

Like reference numerals will be used to refer to like parts from Figure to Figure in the following description of the drawings.

### DETAILED DESCRIPTION OF THE INVENTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. Skilled artisans will recognize the examples provided herein have many useful alternatives and fall within the scope of embodiments of the invention.

Dual Mobility implants are currently being used in 10% of primary and 30% of revision THA. Recently, there has been an increase in the use of cemented dual mobility components in the U.S., especially at the time of revision THA when cementation of dual mobility is preferred into a well fixed shell, highly porous cup at the time of revision or antiprotrusio ring in the same setting. The difficulty lies when extraction of dual mobility cups is needed, for example when dislocation occurs or with infection. Thus, before the present disclosure, an unmet need existed for a dual mobility design that allows partial cementation, providing the same mechanical strength as full cementation, but allowing cementless areas that would ease removal by placing an extraction tool.

A prosthetic system of the invention addresses these challenges by providing a cemented dual mobility acetabular component that allows for partial cementing of the outer surface of the acetabular cup by limiting the outer surface that requires cementing, leaving a gap to then place an extraction tool to remove the acetabular cup. The extraction tool can be size specific, and can be specifically designed to fall into the channels of the space left behind by insert spacers that are removed after cementing has occurred. The extraction tool can come in from opposite directions and allows for connection to a slap hammer that can then extract the acetabular cup.

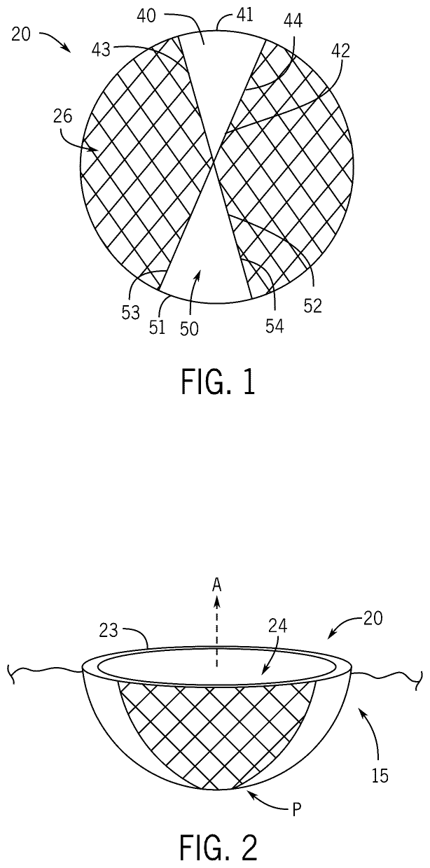

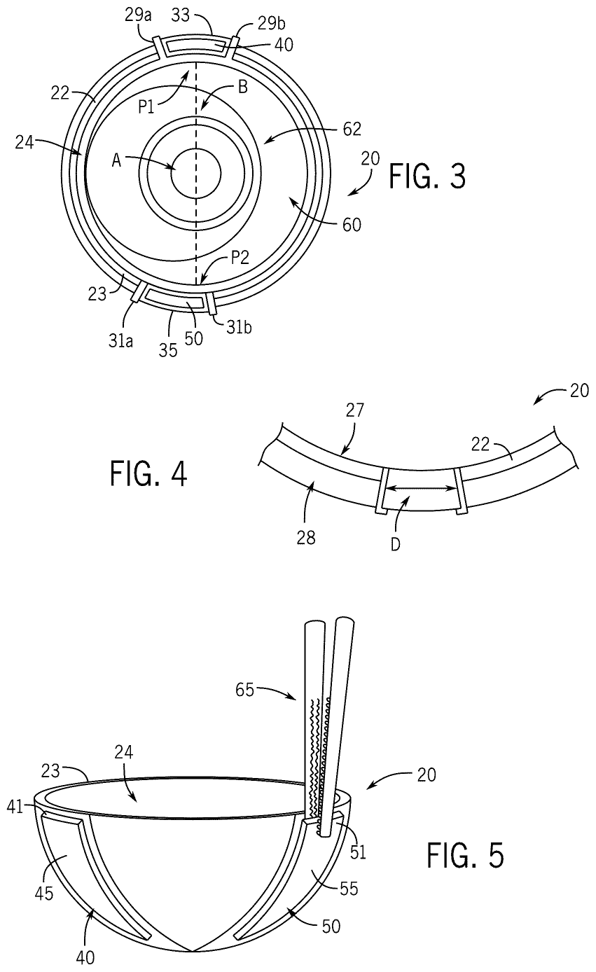

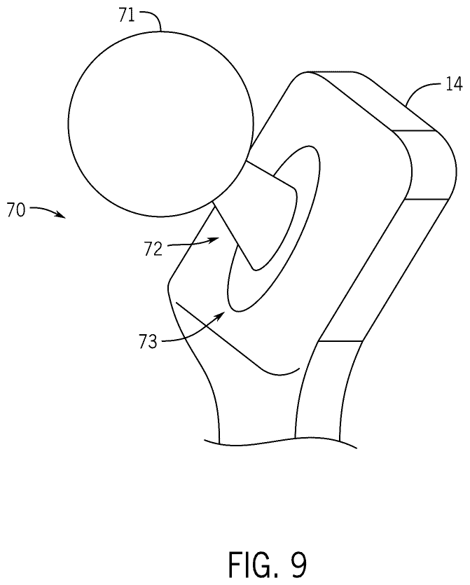

Turning now to FIGS. 1-6 and 9, there is shown one embodiment of a prosthetic system of the invention that provides for motion between a pelvis 15 and a femur 14 of a hip joint of a subject. The prosthetic system includes an acetabular cup 20 including a wall 22 terminating in a rim 23. The wall 22 and the rim 23 define a cavity 24 of the acetabular cup 20. The wall 22 has an outer surface 26 and an inner surface 27 within the cavity 24 of the acetabular cup 20. The acetabular cup 20 has a first pair of spaced apart first projections 29a, 29b extending outwardly from the outer surface 26 of the wall 22 and a second pair of spaced apart second projections 31a, 31b extending outwardly from the outer surface 26 of the wall 22. The first pair of spaced apart first projections 29a, 29b define a first channel 33 that extends to the rim 23, and the second pair of spaced apart second projections 31a, 31b define a second channel 35 that extends to the rim 23. The acetabular cup 20 may be formed from a metal alloy such as a titanium alloy (e.g., titanium-6-aluminum-4-vanadium), a cobalt alloy, a stainless steel alloy or from tantalum; a nonresorbable ceramic such as aluminum oxide or zirconia; a nonresorbable polymeric material such as polyethylene; or a nonresorbable composite material such as a carbon fiber-reinforced polymers (e.g., polysulfone). The prosthetic system includes a first spacer 40 removably positioned in the first channel 33, and a second spacer 50 removably positioned in the second channel 35.

The prosthetic system includes a liner 60 positioned in the cavity 24 of the acetabular cup 20. An additional liner 62 is positioned in an opening of the liner creating a dual mobility hip implant. The liner 60 and the additional liner 62 can comprise a nonresorbable polymeric material, such as polyethylene, preferably ultra-high density polyethylene. The prosthetic system includes a femoral component 70 including a stem 73 dimensioned for insertion in the femur 14, and a head 71 connected to the stem 73 by a neck 72. The head 71 has a first articular surface dimensioned to articulate with a second articular surface of the additional liner 62. When replacing the femoral head with the femoral component 70, the femoral head is surgically removed and the top of the femur 14 and the intramedullary canal of the femur 14 are prepared for receipt of the stem 73. A cement material is typically inserted into the open intramedullary canal and the stem 73 is driven into the canal. Often, the stem is fixed to the femur 14 with a curable acrylic polymer cement such as polymethylmethacrylate. Alternatively, the stem 73 may be fixed to the femur 14 using an interference fit (i.e., “press fit”) without cement. The head 71 of the femoral component 70 can be dimensioned for articulation with the additional liner 62, which provides a second articular surface of the prosthetic system, to ensure proper articulation with the femoral head 71. The femoral component 70 may be formed from a metal alloy such as a titanium alloy (e.g., titanium-6-aluminum-4-vanadium), a cobalt alloy, a stainless steel alloy or from tantalum; a nonresorbable ceramic such as aluminum oxide or zirconia; a nonresorbable polymeric material such as polyethylene; or a nonresorbable composite material such as a carbon fiber-reinforced polymers (e.g., polysulfone).

The first spacer 40 has a first end 41, a second end 42, a side wall 43 and an opposed side wall 44 extending from the first end 41 to the second end 42. The opposed side walls 43, 44 taper inward from the first end 41 to the second end 42. The first end 41 of the first spacer 40 has an indentation 45. A transverse distance D between the spaced apart first projections 29a, 29b decreases when moving from the rim 23 to an intersection point P of a longitudinal axis A of the acetabular cup 20 and the outer surface 26 of the wall 22 of the acetabular cup 20. The first spacer 40 can comprise a polymeric material, such as polyethylene.

The second spacer 50 has a first end 51, a second end 52, a side wall 53 and an opposed side wall 54 extending from the first end 51 to the second end 52. The opposed side walls 53, 54 taper inward from the first end 51 to the second end 52. The first end 51 of the second spacer 50 has an indentation 55. A transverse distance D between the spaced apart second projections 31a, 31b decreases when moving from the rim 23 to an intersection point P of a longitudinal axis A of the acetabular cup 20 and the outer surface 26 of the wall 22 of the acetabular cup 20. The second spacer 50 can comprise a polymeric material, such as polyethylene.

The first spacer 40 and the second spacer 50 can be dimensioned: such that the first spacer 40 and the second spacer 50 cover less than 10% of the surface area of the outer surface 26 of the acetabular cup 20 (providing at least 90% bone cement coverage of the outer surface 26 of the acetabular cup 20), or such that the first spacer 40 and the second spacer 50 cover less than 20% of the surface area of the outer surface 26 of the acetabular cup 20 (providing at least 80% bone cement coverage of the outer surface 26 of the acetabular cup 20), or such that the first spacer 40 and the second spacer 50 cover less than 30% of the surface area of the outer surface 26 of the acetabular cup 20 (providing at least 70% bone cement coverage of the outer surface 26 of the acetabular cup 20), or such that the first spacer 40 and the second spacer 50 cover less than 40% of the surface area of the outer surface 26 of the acetabular cup 20 (providing at least 60% bone cement coverage of the outer surface 26 of the acetabular cup 20), or such that the first spacer 40 and the second spacer 50 cover less than 50% of the surface area of the outer surface 26 of the acetabular cup 20 (providing at least 50% bone cement coverage of the outer surface 26 of the acetabular cup 20), or such that the first spacer 40 and the second spacer 50 cover less than 60% of the surface area of the outer surface 26 of the acetabular cup 20 (providing at least 40% bone cement coverage of the outer surface 26 of the acetabular cup 20), or such that the first spacer 40 and the second spacer 50 cover less than 70% of the surface area of the outer surface 26 of the acetabular cup 20 (providing at least 30% bone cement coverage of the outer surface 26 of the acetabular cup 20). The first spacer 40 and the second spacer 50 preferably have a thickness of at least 2 millimeters.

The acetabular cup 20 has a longitudinal axis A and a transverse axis B perpendicular to the longitudinal axis A. A first intersection point P1 of the transverse axis B and the outer surface 26 of the wall 22 of the acetabular cup 20 is between the spaced apart first projections 29a, 29b. A second intersection point P2 of the transverse axis B and the outer surface 26 of the wall 22 of the acetabular cup 20 is between the spaced apart second projections 31a, 31b.

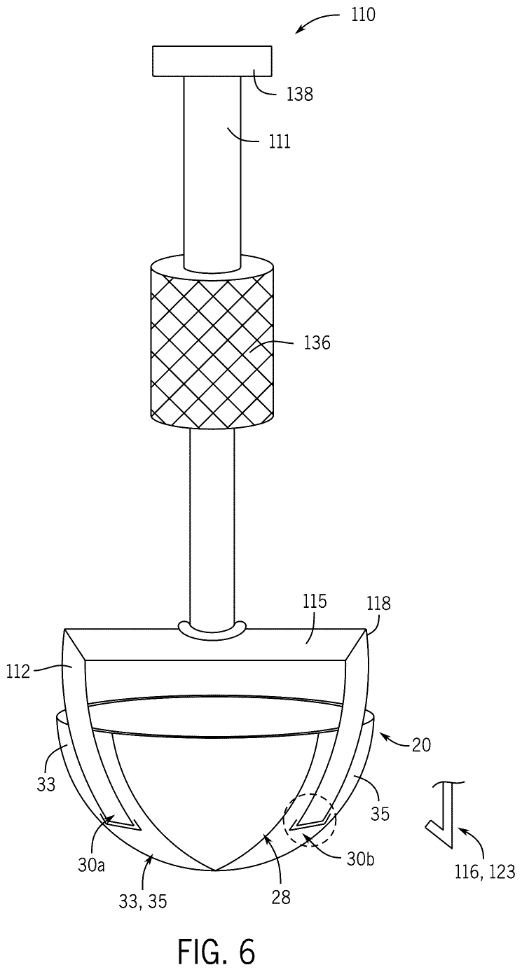

Referring to FIG. 6, the prosthetic system of FIGS. 1-5 and 9 may be provided in a kit for preparing a prosthetic system along with an extraction tool 110 for removing the acetabular cup 20 in a revision surgery. The extraction tool 110 comprises a shaft 111, a first arm 112, and a second arm 118. The first arm 112 and the second arm 118 are attached to the shaft 111 in a spaced apart relationship using a crossbar 115 such that the first arm 112 can engage the outer surface 26 of the wall 22 of the acetabular cup 20 between the first pair of spaced apart first projections 29a, 29b and the second arm 118 can engage the outer surface 26 of the wall 22 of the acetabular cup 20 between the second pair of spaced apart second projections 31a, 31b. The extraction tool 110 can comprise a metal alloy such as a stainless steel alloy.

The first arm 112 includes an inwardly angled first distal end 116 (which is a mirror image of the distal end shown in FIG. 6) dimensioned to engage a first recess 30a in the outer surface 26 of the wall 22 of the acetabular cup 20 for securing the first arm 112 to the acetabular cup 20 when removing the acetabular cup 20. Instead of an angled distal end 116, the distal end 116 can be curved inward.

The second arm 118 includes an inwardly angled second distal end 123 dimensioned to engage a second recess 30b in the outer surface 26 of the wall 22 of the acetabular cup 20 for securing the second arm 118 to the acetabular cup 20 when removing the acetabular cup 20. Instead of an angled distal end 123, the distal end 123 can be curved inward.

The extraction tool 110 further comprises a tubular handle 136 slidable on the shaft 111 and a stop 138 on a proximal end of the shaft 111. The stop 138 transmits force from the handle 136 to the shaft 111 when the handle 136 strikes the stop 138.

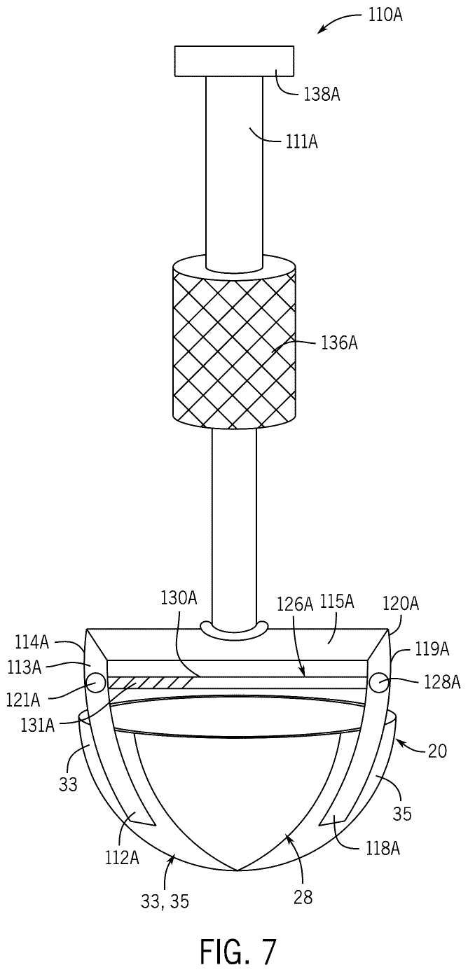

Referring now to FIG. 7, there is shown another non-limiting embodiment of an extraction tool 110A of the present disclosure engaging the acetabular cup 20 of FIGS. 1-5 for removing the acetabular cup 20 in a revision surgery. The extraction tool 110A comprises a shaft 111A, a first arm 112A, and a second arm 118A. The first arm 112A and the second arm 118A are attached to the shaft 111A in a spaced apart relationship using a crossbar 115A such that the first arm 112A can engage the outer surface 26 of the wall 22 of the acetabular cup 20 between the first pair of spaced apart first projections 29a, 29b and the second arm 118A can engage the outer surface 26 of the wall 22 of the acetabular cup 20 between the second pair of spaced apart second projections 31a, 31b. A first hinge 114A connects the first arm 112A to the crossbar 115A which is connected to the shaft 111A. A second hinge 120A connects the second arm 118A to the crossbar 115A which is connected to the shaft 111A. The extraction tool 110A can comprise a metal alloy such as a stainless steel alloy.

The extraction tool 110A comprises an adjustable retention mechanism 126A engaging a first section 113A of the first arm 112A and a second section 119A of the second arm 118A. The retention mechanism 126A provides forces that oppose opening forces exerted on the first arm 112A and the second arm 118A when removing the acetabular cup 20 from the acetabulum in a revision surgery. The retention mechanism 126A comprises a first end section 128A in contact with the first arm 112A and a second end section 130A with threads 131A in contact with threads in an internally threaded hole 121A in the second arm 118A such that the spaced apart relationship of the first arm 112A and the second arm 118A can be varied by rotating the first end section 128A thereby tightening the first arm 112A and the second arm 118A on the acetabular cup 20.

The extraction tool 110A further comprises a tubular handle 136A slidable on the shaft 111A and a stop 138A on a proximal end of the shaft 111A. The stop 138A transmits force from the handle 136A to the shaft 111A when the handle 136A strikes the stop 138A.

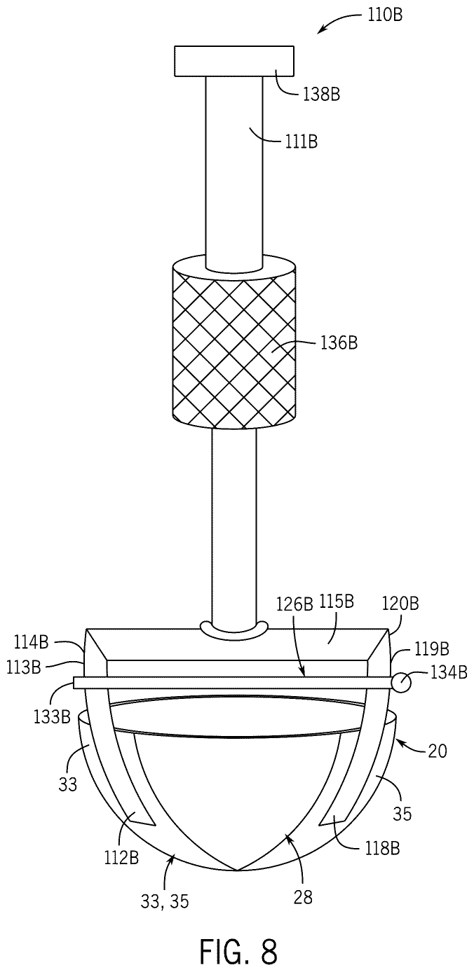

Turning now to FIG. 8, there is shown a side view of yet another non-limiting embodiment of an extraction tool 110B of the present disclosure engaging the acetabular component of FIG. 1 of the present disclosure for removing the acetabular cup 20 in a revision surgery. The extraction tool 110B comprises a shaft 111B, a first arm 112B, and a second arm 118B. The first arm 112B and the second arm 118B are attached to the shaft 111B in a spaced apart relationship using a crossbar 115B such that the first arm 112B can engage the outer surface 26 of the wall 22 of the acetabular cup 20 between the first pair of spaced apart first projections 29a, 29b and the second arm 118B can engage the outer surface 26 of the wall 22 of the acetabular cup 20 between the second pair of spaced apart second projections 31a, 31b. A first hinge 114B connects the first arm 112B to the crossbar 115B which is connected to the shaft 111B. A second hinge 120B connects the second arm 118B to the crossbar 115B which is connected to the shaft 111B. The extraction tool 110B can comprise a metal alloy such as a stainless steel alloy.

The extraction tool 110B comprises an adjustable retention mechanism 126B engaging a first section 113B of the first arm 112B and a second section 119B of the second arm 118B. The retention mechanism 126B provides forces that oppose opening forces exerted on the first arm 112B and the second arm 118B when removing the acetabular cup 20 from the acetabulum in a revision surgery. The retention mechanism 126B comprises a band 133B in contact with the first arm 112B and the second arm 118B, and a captive screw 134B engaging with threads on the band 133B such the band 133B can tighten around the first arm 112B and the second arm 118B by rotating the screw 134B thereby tightening the first arm 112B and the second arm 118B on the acetabular cup 20.

The extraction tool 110B further comprises a tubular handle 136B slidable on the shaft 111B and a stop 138B on a proximal end of the shaft 111B. The stop 138B transmits force from the handle 136B to the shaft 111B when the handle 136B strikes the stop 138B.

Looking at FIGS. 1-9, the present invention provides a method for replacing at least a part of a hip joint of a subject. The method comprises: (a) providing an acetabular component including: (i) the acetabular cup 20 including the wall 22 terminating in the rim 23, the wall 22 and the rim 23 defining the cavity 24 of the acetabular cup 20, the wall 22 having the outer surface 26 and the inner surface 27 within the cavity 24 of the acetabular cup 20, the acetabular cup 20 having the first pair of spaced apart first projections 29a, 29b extending outwardly from the outer surface 26 of the wall 22 and the second pair of spaced apart second projections 31a, 31b extending outwardly from the outer surface 26 of the wall 22, the first pair of spaced apart first projections 29a, 29b defining the first channel 33 that extends to the rim 23, and the second pair of spaced apart second projections 31a, 31b defining the second channel 35 that extends to the rim 23, (ii) the first spacer 40 removably positioned in the first channel 33, and (iii) the second spacer 50 removably positioned in the second channel 35; (b) preparing a pelvis of the hip joint for receiving the acetabular component; (c) applying bone cement 28 to the outer surface 26 of the wall 22 of the acetabular cup 20; (d) implanting the acetabular component in the pelvis; (e) removing the first spacer 40 from the first channel 33; (f) removing the second spacer 50 from the second channel 35; (g) positioning the liner 60 in the cavity 24 of the acetabular cup 20; (h) positioning the additional liner 62 in an opening of the liner 60; (i) preparing the femur of the hip joint for receiving the stem 73 of the femoral component 70 in a canal in the femur; (j) implanting the stem 73 of the femoral component 70 in the canal in the femur; and (k) positioning the head 71 of the femoral component 70 to articulate with an articular surface of the additional liner 62.

In one embodiment of the method for replacing at least a part of a hip joint of a subject, step (c) comprises applying the bone cement 28 to the outer surface 26 of the wall 22 of the acetabular cup 20 such that the bone cement 28 does not contact the first spacer 40 and the second spacer 50.

In one embodiment of the method, step (c) comprises applying the bone cement 28 to the outer surface 26 of the wall 22 of the acetabular cup 20 such that the bone cement 28 has a thickness that is equal to or less than a height of at least one of the first projections 29a, 29b and the second projections 31a, 31b. In one embodiment of the method, step (c) comprises applying the bone cement 28 to the outer surface 26 of the wall 22 of the acetabular cup 20 such that the bone cement 28 has a thickness that is equal to or less than a height of all of the first projections 29a, 29b and the second projections 31a, 31b. In one embodiment of the method, step (e) comprises removing the first spacer 40 from the first channel 33 using a clamp 65 that engages the indentation 45 in the first end 41 of the first spacer 40, and step (f) comprises removing the second spacer 50 from the second channel 35 using the clamp 65 to engage the indentation 55 in the first end 51 of the second spacer 50.

Looking at FIGS. 1-9, the present invention also provides a method of revising a total hip arthroplasty. The method comprises: (a) providing the extraction tool 110, comprising the shaft 111, the first arm 112, and the second arm 118, the first arm 112 and the second arm 118 being attached to the shaft 111 in a spaced apart relationship; (b) positioning the first arm 112 and the second arm 118 of the extraction tool 110 in contact with an existing acetabular component implanted in an acetabulum, wherein the existing acetabular component includes: (i) the acetabular cup 20 including the wall 22 terminating in the rim 23, the wall 22 and the rim 23 defining the cavity 24 of the acetabular cup 20, the wall 22 having the outer surface 26 and the inner surface 27 within the cavity 24 of the acetabular cup 20, the acetabular cup 20 having the first pair of spaced apart first projections 29a, 29b extending outwardly from the outer surface 26 of the wall 22 and the second pair of spaced apart second projections 31a, 31b extending outwardly from the outer surface 26 of the wall 22, the first pair of spaced apart first projections 29a, 29b defining the first channel 33 that extends to the rim 23, and the second pair of spaced apart second projections 31a, 31b defining the second channel 35 that extends to the rim 23; (c) engaging the first arm 112 of the extraction tool 110 with the outer surface 26 of the wall 22 of the acetabular cup 20 between the first pair of spaced apart first projections 29a, 29b; (d) engaging the second arm 118 of the extraction tool 110 with the outer surface 26 of the wall 22 of the acetabular cup 20 between the second pair of spaced apart second projections 31a, 31b; and (e) applying a force to the extraction tool 110 such that the existing acetabular component is removed from the acetabulum.

In one embodiment of the method of revising a total hip arthroplasty of a subject, the extraction tool 110 further comprises the tubular handle 136 slidable on the shaft 111 and the stop 138 on the proximal end of the shaft 111, and step (e) comprises applying the force to the extraction tool 110 by striking the stop 138 with the handle 136 thereby transmitting force from the handle 136 to the shaft 111 when the handle 136 strikes the stop 138 such that the acetabular component is removed from the acetabulum. In another embodiment of the method for replacing at least a part of a hip joint of a subject, step (e) comprises applying the force to the extraction tool 110 by twisting the extraction tool 110 such that the acetabular component is removed from the acetabulum.

In one embodiment of the method of revising a total hip arthroplasty, the first arm 112 includes the angled or curved first distal end 116 dimensioned to engage the first recess 30a in the outer surface 26 of the wall 22 of the acetabular cup 20, the second arm 118 includes the angled or curved second distal end 123 dimensioned to engage the second recess 30b in the outer surface 26 of the wall 22 of the acetabular cup 20, step (c) further comprises engaging the first arm 112 of the extraction tool 110 with the first recess 30a in the outer surface 26 of the wall 22 of the acetabular cup 20, and step (d) further comprises engaging the second arm 118 of the extraction tool 110 with the second recess 30b in the outer surface 26 of the wall 22 of the acetabular cup 20.

The extraction tool 110A or the extraction tool 110B can be used in other embodiments of the method of revising a total hip arthroplasty. In one of these embodiments of the method, step (d) further comprises applying a retaining force on the first arm 112A and the second arm 118A using adjustable retention mechanism 126A, the retaining force opposing opening forces exerted on the first arm 112A and the second arm 118A. In another of these embodiments of the method, step (d) further comprises applying a retaining force on the first arm 112B and the second arm 118B using adjustable retention mechanism 126B, the retaining force opposing opening forces exerted on the first arm 112B and the second arm 118B.

In one embodiment of the method of revising a total hip arthroplasty of a subject, step (e) comprises applying the force to the extraction tool 110 or the extraction tool 110A or the extraction tool 110B by striking the stop 138 or 138A or 138B with the handle 136 or 136A or 136B thereby transmitting force from the handle 136 or 136A or 136B to the shaft 111 or 111A or 111B when the handle 136 or 136A or 136B strikes the stop 138 or 138A or 138B such that the acetabular component is removed from the acetabulum. In another embodiment of the method for replacing at least a part of a hip joint of a subject, step (e) comprises applying the force to the extraction tool 110 or the extraction tool 110A or the extraction tool 110B by twisting the extraction tool 110 or the extraction tool 110A or the extraction tool 110B such that the acetabular component is removed from the acetabulum.

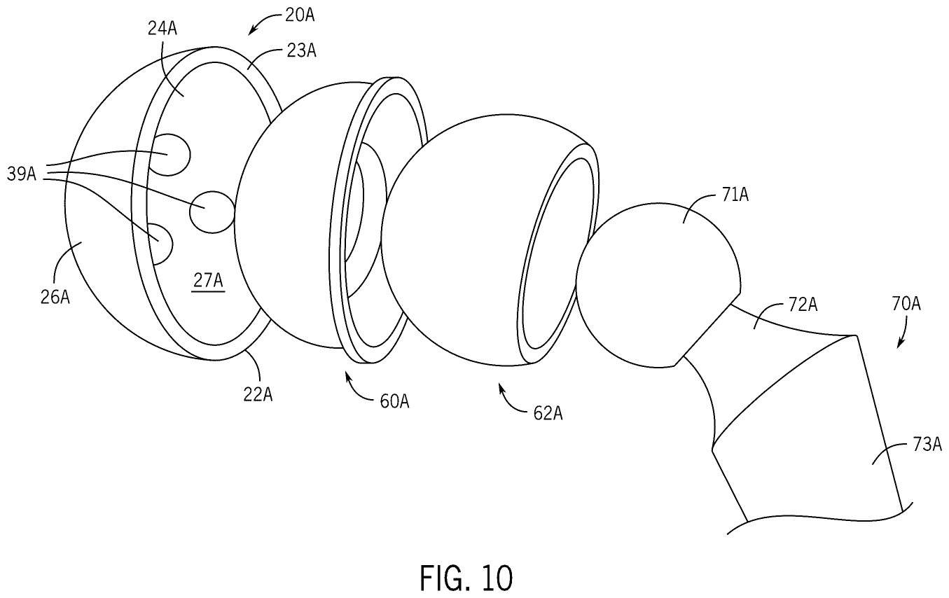

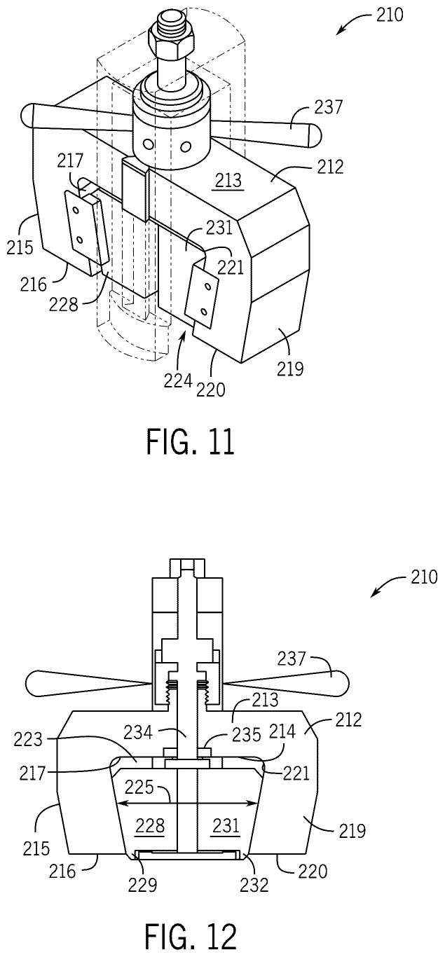

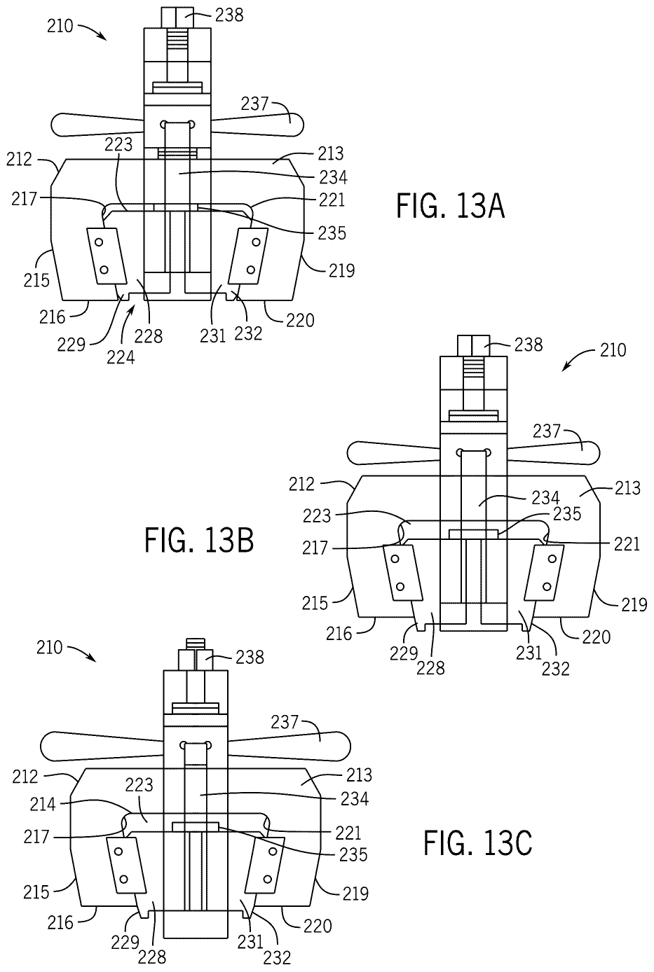

Referring now to FIGS. 11, 12, 13A, 13B, and 13C, there is shown still another non-limiting embodiment of an extraction tool 210 of the present disclosure engaging the acetabular component of FIG. 1 of the present disclosure for removing the acetabular cup 20 in a revision surgery. The extraction tool 210 includes a frame 212 comprising a support 213, a first arm 215 having a first distal end 216 and an inner surface 217, and a second arm 219 having a second distal end 220 and an inner surface 221. The support 213, the first arm 215, and the second arm 219 define an interior space 223 and an opening 224 of the frame 212. The extraction tool 210 includes a first block 228 moveable with respect to the frame 212 having a first distal projection 229 and a second block 231 moveable with respect to the frame 212 having a second distal projection 232. The extraction tool 210 includes a push rod 234 having a flange 235, and a handle 237 surrounding and engaging the push rod 234. A nut 238 can secure the handle 237 to the push rod 234.

In one embodiment of the extraction tool 210, the first block 228 and the second block 231 are located in a spaced apart relationship in the interior space 223 of the frame 212 such that the first block 228 can engage the outer surface 26 of the wall 22 of the acetabular cup 20 and the second block 231 can engage the outer surface 26 of the wall 22 of the acetabular cup 20. The first arm 215 and the second arm 219 are angled inward with respect to the support 213 such that a distance 225 between the first arm 215 and the second arm 219 decreases from an inner surface 214 of the support 213 towards the opening 224 defined by the first distal end 216 of the first arm 215 and a second distal end 220 of the second arm 219. The first block 228 contacts the inner surface 217 of the first arm 215, and the second block 231 contacts the inner surface 221 of the second arm 219 such that the first block 228 and the second block 231 move laterally toward each other when the first block 228 and the second block 231 move toward the opening 224.

The flange 235 of the push rod 234 contacts the first block 228 and the second block 231 and the flange 235 moves the first block 228 and the second block 231 toward the opening 224 when the push rod 234 is moved downward toward the opening 224. The handle 237 engages the push rod 234 for translating the push rod 234 towards the first block 228 and the second block 231 and for moving the first block 228 and the second block 231 toward the opening 224. When the first block 228 and the second block 231 move toward the opening 224, the first distal projection 229 and the second distal projection 232 move toward each other for engaging the outer surface 26 of the wall 22 of the acetabular cup 20.

Looking at FIGS. 11, 12, 13A, 13B, and 13C, the present invention also provides a method of revising a total hip arthroplasty. In one embodiment, the method comprises: (a) providing the extraction tool 210; (b) positioning the first block 228 and the second block 231 of the extraction tool 210 in contact with an existing acetabular component implanted in an acetabulum, wherein the acetabular component includes: (i) an acetabular cup 20 including a wall 22 terminating in a rim 23, the wall 22 and the rim 23 defining a cavity 24 of the acetabular cup 20, the wall 22 having an outer surface 26 and an inner surface 27 within the cavity 24 of the acetabular cup 20, the acetabular cup 20 having a first pair of spaced apart first projections 29a, 29b extending outwardly from the outer surface 26 of the wall 22 and a second pair of spaced apart second projections 31a, 31b extending outwardly from the outer surface 26 of the wall 22, the first pair of spaced apart first projections 29a, 29b defining a first channel 33 that extends to the rim 23, and the second pair of spaced apart second projections 31a, 31b defining a second channel 35 that extends to the rim 23; (c) engaging the first block 228 of the extraction tool 210 with the outer surface 26 of the wall 22 of the acetabular cup 20 between the first pair of spaced apart first projections 29a, 29b; (d) engaging the second block 231 of the extraction tool 210 with the outer surface 26 of the wall 22 of the acetabular cup 20 between the second pair of spaced apart second projections 31a, 31b; and (e) applying a force to the extraction tool 210 such that the existing acetabular component is removed from the acetabulum. In one embodiment of the method, step (e) comprises applying a pulling force to the extraction tool 210. In one embodiment of the method, step (e) comprises applying the force to the extraction tool 210 by twisting the extraction tool 210 such that the existing acetabular component is removed from the acetabulum.

In one embodiment of the method, the frame 212 of the extraction tool 210 comprises the support 213, the first arm 215, and the second arm 219, the first arm 215 and the second arm 219 being attached to the support 213 in a spaced apart relationship thereby defining the interior space 223 of the frame 212.

In one embodiment of the method, one or both of the first arm 215 and the second arm 219 of the extraction tool 210 are angled with respect to the support 213 such that the distance 225 between the first arm 215 and the second arm 219 decreases from the inner surface 214 of the support 213 towards the opening 224 defined by the first distal end 216 of the first arm 215 and the second distal end 220 of the second arm 219, the first block 228 contacts the inner surface 217 of the first arm 215, the second block 231 contacts the inner surface 221 of the second arm 219, and the first block 228 and the second block 231 move toward each other when the first block 228 and the second block 231 move toward the opening 224. In one embodiment of the method, the extraction tool 210 comprises the push rod 243 having the flange 235 that contacts the first block 228 and the second block 231 and moves the first block 228 and the second block 231 toward the opening 224. The first block 228 includes the first distal projection 229 for engaging the outer surface 26 of the wall 22 of the acetabular cup 20; and the second block 231 includes the second distal projection 232 for engaging the outer surface 26 of the wall 22 of the acetabular cup 20.

Turning now to FIG. 10, there is shown another embodiment of a prosthetic system of the invention that provides for motion between a pelvis and a femur of a hip joint of a subject. The prosthetic system includes an acetabular cup 20A including a wall 22A terminating in a rim 23A. The wall 22A and the rim 23A define a cavity 24A of the acetabular cup 20A. The wall 22A has an outer surface 26A and an inner surface 27A within the cavity 24A of the acetabular cup 20A. The acetabular cup 20A includes throughholes 39A. The acetabular cup 20A may be formed from a metal alloy such as a titanium alloy (e.g., titanium-6-aluminum-4-vanadium), a cobalt alloy, a stainless steel alloy or from tantalum; a nonresorbable ceramic such as aluminum oxide or zirconia; a nonresorbable polymeric material such as polyethylene; or a nonresorbable composite material such as a carbon fiber-reinforced polymers (e.g., polysulfone).

The prosthetic system includes a liner 60A positioned in the cavity 24A of the acetabular cup 20A. An additional liner 62A is positioned in an opening of the liner 60A creating a dual mobility hip implant. The liner 60A may be formed from a metal alloy such as a titanium alloy (e.g., titanium-6-aluminum-4-vanadium), a cobalt alloy, a stainless steel alloy or from tantalum; a nonresorbable ceramic such as aluminum oxide or zirconia; a nonresorbable polymeric material such as polyethylene; or a nonresorbable composite material such as a carbon fiber-reinforced polymers (e.g., polysulfone). The additional liner 62A can comprise a nonresorbable polymeric material, such as polyethylene, preferably ultra-high density polyethylene. The prosthetic system includes a femoral component 70A including a stem 73A dimensioned for insertion in the femur, and a head 71A connected to the stem 73A by a neck 72A. The head 71A has a first articular surface dimensioned to articulate with a second articular surface of the additional liner 62A. When replacing the femoral head with the femoral component 70A, the femoral head is surgically removed and the top of the femur and the intramedullary canal of the femur are prepared for receipt of the stem 73A. A cement material is typically inserted into the open intramedullary canal and the stem 73A is driven into the canal. Often, the stem is fixed to the femur with a curable acrylic polymer cement such as polymethylmethacrylate. Alternatively, the stem 73A may be fixed to the femur using an interference fit (i.e., “press fit”) without cement. The head 71A of the femoral component 70A can be dimensioned for articulation with the additional liner 62A, which provides a second articular surface of the prosthetic system, to ensure proper articulation with the femoral head 71A. The femoral component 70A may be formed from a metal alloy such as a titanium alloy (e.g., titanium-6-aluminum-4-vanadium), a cobalt alloy, a stainless steel alloy or from tantalum; a nonresorbable ceramic such as aluminum oxide or zirconia; a nonresorbable polymeric material such as polyethylene; or a nonresorbable composite material such as a carbon fiber-reinforced polymers (e.g., polysulfone).

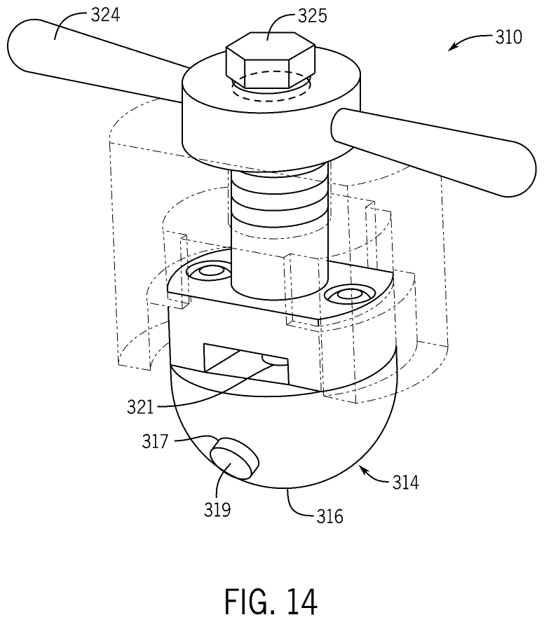

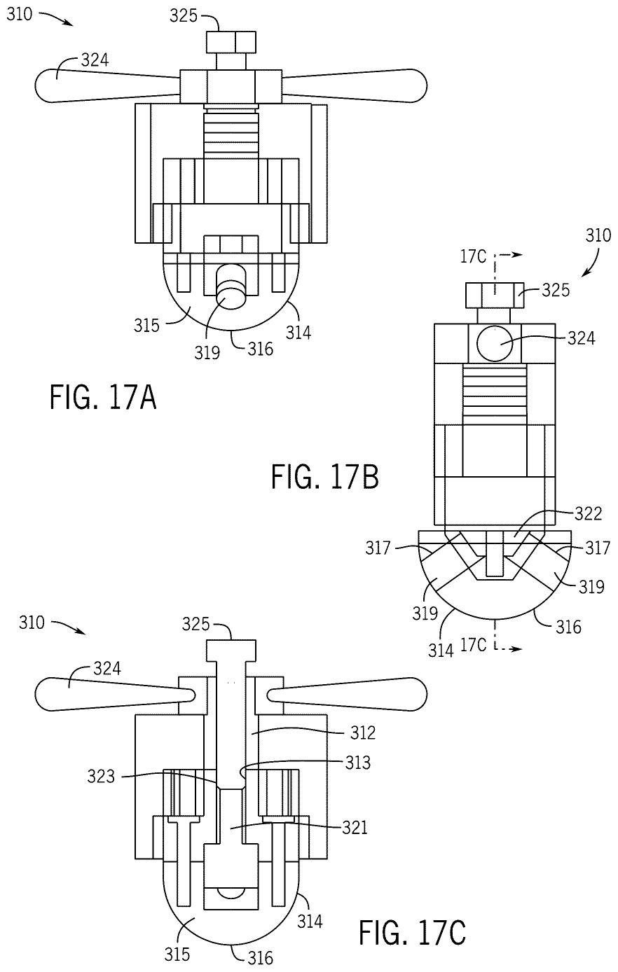

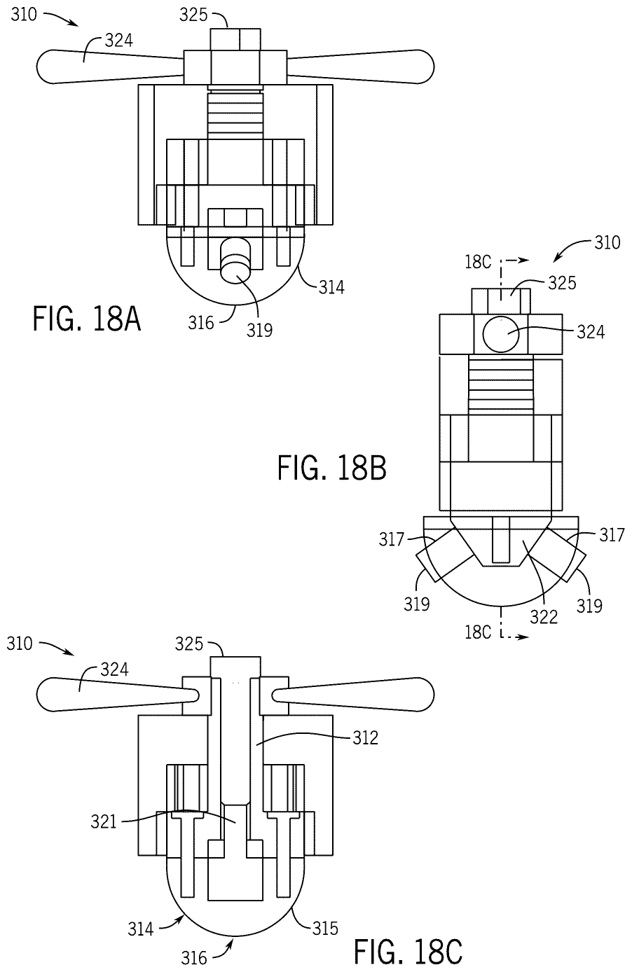

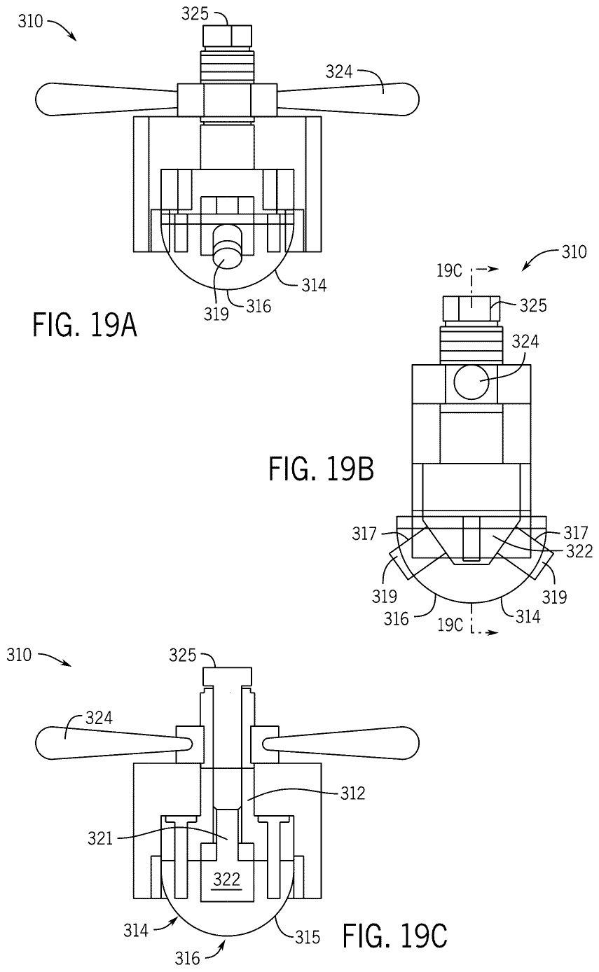

Referring now to FIGS. 14, 15A, 15B, 16A, 16B, 16C, 16D, 17A, 17B, 17C, 18A, 18B, 18C, 19A, 19B, and 19C, there is shown still another non-limiting embodiment of an extraction tool 310 of the present disclosure engaging the acetabular component of FIG. 10 of the present disclosure for removing the acetabular cup 20A in a revision surgery. Looking at FIGS. 14, 17A, 17B, 17C, 18A, 18B, 18C, 19A, 19B, and 19C, the extraction tool 310 includes a frame 312 having internal threads 313, and a cap 314 having a wall 315 having a distal surface 316 and holes 317. Moveable pins 319 are located in the holes 317. The extraction tool 310 includes a shaft 321 having a distal end section 322 and external threads 323, and a handle 324 that engages the shaft 321. A nut 325 can secure the handle 324 to the shaft 321.

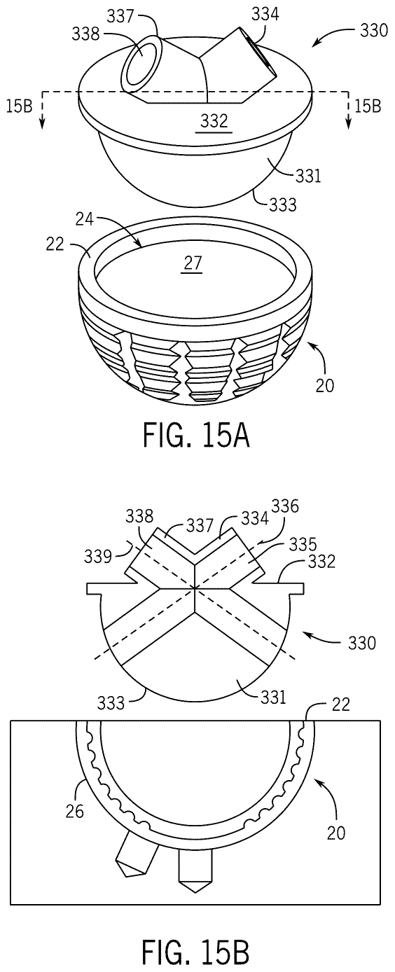

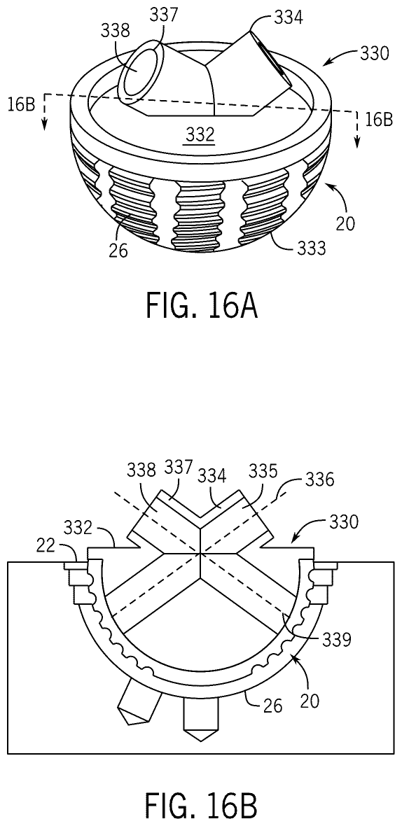

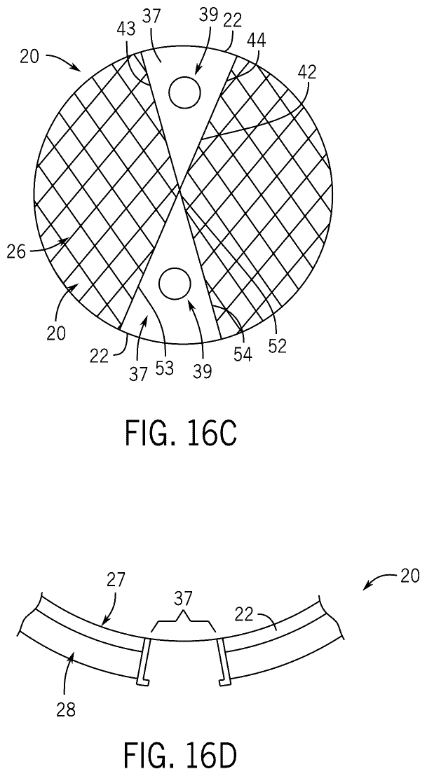

Turning to FIGS. 15A, 15B, 16A, 16B, 16C, and 16D, the extraction tool 310 includes a drill guide 330 having a body 331 having a proximal surface 332 and a distal surface 333. In one embodiment, the distal surface 333 of the drill guide 330 includes a spherical portion. The drill guide 330 has a first tubular wall 334 defining a first section of a first passageway 335, and a second section of the first passageway 335 extends through the wall 334. The first passageway 335 has a first longitudinal axis 336. The drill guide 330 has a second tubular wall 337 defining a first section of a second passageway 338, and a second section of the second passageway 338 extends through the wall 334. The second passageway 338 has a second longitudinal axis 339. The first longitudinal axis 336 of the first passageway 335 and the second longitudinal axis 339 of the second passageway 338 are in a reference plane in the page of the drawing sheet. In one embodiment, the first longitudinal axis 336 and the second longitudinal axis 339 intersect.

In one embodiment of the extraction tool 310, the drill guide 330 is dimensioned such that the distal surface 333 of the drill guide 330 can engage the inner surface 27 within the cavity 24 of the acetabular cup 20. Then, the first passageway 335 and second passageway 338 of the drill guide 330 can direct a boring instrument (e.g., a drill) into the wall of the acetabular cup 20 for creating throughholes 39 in the wall 22 of the acetabular cup 20. See FIG. 16C. In one embodiment, the throughholes 39 can be created in a thinned section 37 of the wall 22 of the acetabular cup 20 located where the first spacer 40 and the second spacer 50 have been removed. See FIGS. 16C and 16D.

The shaft 321 of the extraction tool 310 is dimensioned to move each of the pins 319 into an associated mounting hole 39 of the acetabular cup 20 when the distal surface 316 of the cap 314 engages the inner surface 27 within the cavity 24 of the acetabular cup 20. Alternatively, the acetabular cup 20 can be manufactured with the throughholes 39 thereby eliminating the need for later creation of the throughholes 39. The handle 324 engages the shaft 321 for translating the shaft 321 towards the cap 314 and moving each of the pins 319 into an associated throughhole 39 of the acetabular cup 20 when the distal surface 316 of the cap 314 engages the inner surface 27 within the cavity 24 of the acetabular cup 20. In one embodiment, the shaft 321 includes a frustoconical distal end section 322 for moving each of the pins 319 into an associated throughhole 39 of the acetabular cup 20. In one embodiment, the shaft 321 includes external threads 323 that engage internal threads 313 of the frame 312 for moving the shaft 321 with respect to the frame 312.

Looking at FIGS. 14, 15A, 15B, 16A, 16B, 16C, 16D, 17A, 17B, 17C, 18A, 18B, 18C, 19A, 19B, and 19C, the present invention also provides a method of revising a total hip arthroplasty. One embodiment of the method comprises: (a) providing the extraction tool 310 comprising: (i) the frame 312, (ii) the cap 314 secured to the frame 312, the cap 314 having the wall 315 with holes 317, (iii) the pin 319 in each hole 317 of the cap 314, and (iv) the shaft 321 dimensioned to move each of the pins 319 outward in its associated hole 317 of the cap 314; (b) positioning the distal surface 316 of the cap 314 in contact with an existing acetabular component implanted in an acetabulum, wherein the acetabular component includes: (i) an acetabular cup 20 including a wall 22 terminating in a rim 23, the wall 22 and the rim 23 defining a cavity 24 of the acetabular cup 20, the wall 22 having an outer surface 26 and an inner surface 27 within the cavity 24 of the acetabular cup 20, the acetabular cup 20 having throughholes 39 in the wall 22 of the acetabular cup 20; (c) translating the shaft 321 of the extraction tool 310 to move each of the pins 319 into an associated throughhole 39 of the acetabular cup 20; and (d) applying a force to the extraction tool 310 such that the existing acetabular component is removed from the acetabulum.

In one embodiment of the method, step (d) comprises applying the force to the extraction tool 310 by twisting the extraction tool 310 such that the existing acetabular component is removed from the acetabulum. In one embodiment of the method, step (d) comprises applying a pulling force to the extraction tool 310 such that the existing acetabular component is removed from the acetabulum. In one embodiment of the method, step (b) comprises directing a boring instrument into the wall 22 of the acetabular cup 20 for creating the throughholes 39 in the wall of the acetabular cup 20. In one embodiment of the method, step (b) further comprises engaging the distal surface 333 of the drill guide 330 with the inner surface 27 within the cavity 24 of the acetabular cup 20, and directing the boring instrument into the wall 22 of the acetabular cup 20 for creating the throughholes 39 in the wall 22 of the acetabular cup 20. In one embodiment of the method, the drill guide 330 includes at least two passageways 335, 338 for directing the boring instrument into the wall 22 of the acetabular cup 20 for creating the throughholes 39 in the wall 22 of the acetabular cup 20.

### Example

The following Example has been presented in order to further illustrate the invention and is not intended to limit the invention in any way. The statements provided in the Example are presented without being bound by theory.

Design & Fabrication: To inform the design of an acetabular cup extraction tool, we conducted a series of mechanical tests on cemented dual mobility hip implants. These tests evaluated the properties of bone cement in the acetabular cup and determined the optimal directionality of forces for efficient removal. Additionally, we tested spacers that provide a 25% or 50% bone cement reduction to assess their effectiveness in facilitating removal with an extraction tool. It is believed that the bond failure during removal is always the tensile strength of the bone cement.

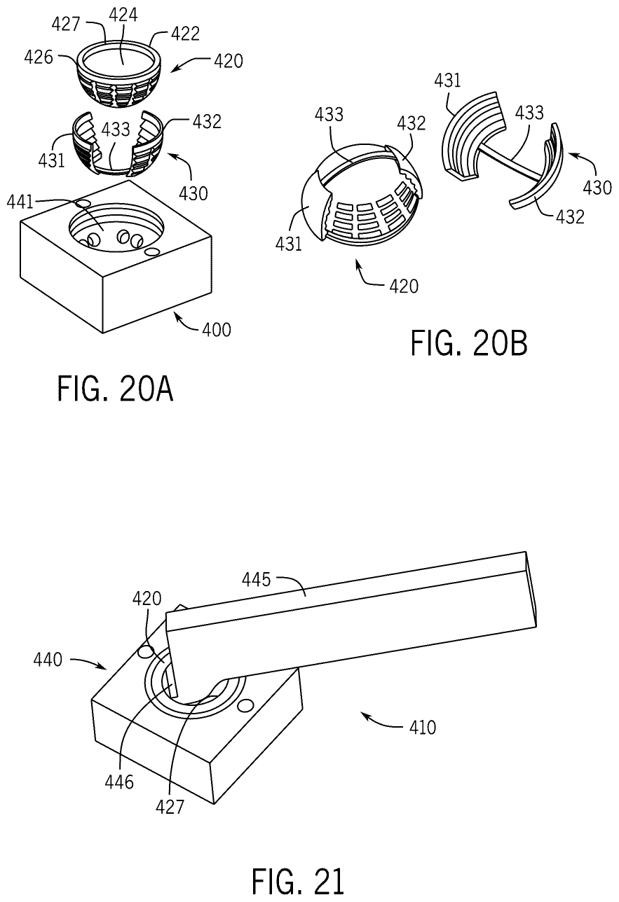

In the absence of a standard test or benchmark for the fixation strength of a bond between cement and an acetabular cup, we developed our own test system. Referring now FIGS. 20A, 20B, and 21, a test system 410 includes an acetabular cup 420 having a wall 422 defining a cavity 424 of the acetabular cup 420. The wall 422 has an outer surface 426 and an inner surface 427 within the cavity 424 of the acetabular cup 420. A test fixture 430 includes a first bone cement mask 431, a second bone cement mask 432, and a connecting bar 433 that attaches the first bone cement mask 431 and the second bone cement mask 432. The test system 410 includes a base 440 having a well 441 formed therein. The test system 410 includes a lever 445 terminating in a projection 446 that engages a notch in the inner surface 427 within the cavity 424 of the acetabular cup 420. The lever 445 rides on a fulcrum and a downward force is applied to an end of the lever 445 opposite the projection 446 during testing.

The first bone cement mask 431 and the second bone cement mask 432 act as spacers that provide a predetermined bone cement reduction from 100% coverage on the outer surface 426 of the acetabular cup 420 when the acetabular cup 420 is cemented in the well 441 of the base 440 for testing. Specifically, the first bone cement mask 431 and the second bone cement mask 432 can be dimensioned with different top and bottom arc lengths to provide a predetermined bone cement reduction from 100% coverage on the outer surface 426 of the acetabular cup 420 when the acetabular cup 420 is cemented in the well 441 of the base 440 for testing. After the acetabular cup 420 comprising 3D printed titanium was cemented with methylmethacrylate bone cement in the well 441 of the base 440 for testing with a predetermined bone cement reduction from 100% coverage, the projection 446 of the lever 445 was engaged with the notch in the inner surface 427 within the cavity 424 of the acetabular cup 420 and the downward force on the lever 445 at the release point of the acetabular cup 420 from the well 441 of the base 440 was recorded. For comparative purposes, the force recorded for each of a predetermined bone cement reduction from 100% coverage can be compared to a control test sample at 100% coverage on the outer surface 426 of the acetabular cup 420.

Results are shown below in Table 1.

TABLE 1

| Cement/Liner Ratio | Sample | Break Force (N) | Max Force (N) |
|---|---|---|---|
| 100/0 (Control) | 1 | 1669.26 | 3711.69 |
| | 2 | 2439.65 | 5097.38 |
| | 3 | 11896.75 | 11896.75 |
| | 4 | 2046.84 | 5015.51 |
| | 5* | 13133.93 | 13133.93 |
| | Mean | 5335.22 | 6901.94 |
| | Std. Dev. | 5695.49 | 4380.77 |
| 75/25 (25% bone cement | 1 | 3493.19 | 5520.26 |
| reduction) | 2 | 4066.96 | 4964.06 |
| | 3 | 1911.57 | 6052.51 |
| | Mean | 3157.24 | 5512.28 |
| | Std. Dev. | 1116.27 | 544.27 |
| 50/50 (50% bone cement | 1 | 3469.27 | 3469.27 |
| reduction) | 2 | 3678.84 | 3678.84 |
| | 3 | 1065.31 | 3330.37 |
| | Mean | 2737.80 | 3492.82 |
| | Std. Dev. | 1452.21 | 175.43 |

*Cement fixation never failed, lever deformed the cup liner

The cup design with 50% cement requires much more force to remove than other published results. This supports our theory that 50% cementing of the cup is enough to obtain a biomechanical stable construct, and thus would allow fixation in vivo, without risk of dissociation. Certain published studies (see D'Apuzzo et al., “Assessment of Damage on a Dual Mobility Acetabular System”, J. Arthroplasty 2016 August; 31 (8): 1828-1835; Buller et al., “No difference in force required for intraprosthetic dislocation of mixed manufacturer vs same manufacturer dual mobility articulations”, The Journal of Arthroplasty 35.2 2020:597-602; and Tradonsky et al., “Performance characteristics of two-piece acetabular cups” Scientific Exhibit, AAOS 1991:246) exist in which the data provided is in Ultimate Torque (Nm) and our data collected in this Example is in Break Force (N) (see Table 1) which can be converted to torque by measuring the length of the lever 445. Once converted to torque, we can directly compare to historical acetabular cups in published data. If these historical values fall below the 100% cemented value then we can determine a % bone cement that corresponds to this and test tool designs that work on the lever out principle at this lower value.

These results support our design concept of an extraction tool that would be able to break that cement bond if needed to remove the acetabular cup. The insights gained from these experiments, combined with our design input, are used to develop an extraction tool that can safely and efficiently remove cemented dual mobility hip implants, addressing a critical unmet clinical need.

Cemented dual mobility hip implants pose a significant challenge for surgeons due to their difficulty in removal. This limitation can hinder treatment options for patients experiencing implant failure or infection. To address this issue, there is a need for new cemented designs that incorporate instrumentation to facilitate easier removal, thereby improving patient outcomes and surgical efficiency. The present invention meets these needs by providing a hip joint prosthetic system having an acetabular component and an extraction tool for use with the acetabular component.

In light of the principles and example embodiments described and illustrated herein, it will be recognized that the example embodiments can be modified in arrangement and detail without departing from such principles. Also, the foregoing discussion has focused on particular embodiments, but other configurations are also contemplated. In particular, even though expressions such as “in one embodiment”, “in other embodiments”, “in another embodiment”, or the like are used herein, these phrases are meant to generally reference embodiment possibilities, and are not intended to limit the invention to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another, unless indicated otherwise.

Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

1. A prosthetic system for providing motion between a pelvis and a femur of a hip joint of a subject, the system comprising:
an acetabular cup including a wall terminating in a rim, the wall and the rim defining a cavity of the acetabular cup, the wall having an outer surface and an inner surface within the cavity of the acetabular cup, the acetabular cup having a first pair of spaced apart first projections extending outwardly from the outer surface of the wall and a second pair of spaced apart second projections extending outwardly from the outer surface of the wall, the first pair of spaced apart first projections defining a first channel that extends to the rim, and the second pair of spaced apart second projections defining a second channel that extends to the rim;
a first spacer removably positioned in the first channel; and
a second spacer removably positioned in the second channel.

2. The prosthetic system of claim 1 wherein:
a transverse distance between at least one of the spaced apart first projections and the spaced apart second projections decreases when moving from the rim to an intersection point of a longitudinal axis of the acetabular cup and the outer surface of the wall of the acetabular cup.

3. The prosthetic system of claim 2 wherein:
at least one of the first spacer and the second spacer includes a first end, a second end, and opposed side walls extending from the first end to the second end, the opposed side walls tapering inward from the first end to the second end.

4. The prosthetic system of claim 3 wherein:
the first end has an indentation.

5. The prosthetic system of claim 1 further comprising:
a liner positioned in the cavity of the acetabular cup.

6. The prosthetic system of claim 5 further comprising:
a femoral component including a stem dimensioned for insertion in the femur, and a head connected to the stem, the head having a first articular surface dimensioned to articulate with a second articular surface of the liner.

7. The prosthetic system of claim 5 further comprising:
an additional liner positioned in an opening of the liner.

8. The prosthetic system of claim 7 further comprising:
a femoral component including a stem dimensioned for insertion in the femur, and a head connected to the stem, the head having a first articular surface dimensioned to articulate with a second articular surface of the additional liner.

9. The prosthetic system of claim 1 wherein:
the first spacer and the second spacer are dimensioned such that the first spacer and the second spacer cover less than 60% of a surface area of the outer surface of the acetabular cup.

10. The prosthetic system of claim 1 wherein:
the acetabular cup has a longitudinal axis and a transverse axis perpendicular to the longitudinal axis,
a first intersection point of the transverse axis and the outer surface of the wall of the acetabular cup is between the spaced apart first projections, and
a second intersection point of the transverse axis and the outer surface of the wall of the acetabular cup is between the spaced apart second projections.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. A kit for preparing a prosthetic system for providing motion between a pelvis and a femur of a hip joint of a subject, the kit comprising:
an acetabular cup including a wall terminating in a rim, the wall and the rim defining a cavity of the acetabular cup, the wall having an outer surface and an inner surface within the cavity of the acetabular cup, the acetabular cup having a first pair of spaced apart first projections extending outwardly from the outer surface of the wall and a second pair of spaced apart second projections extending outwardly from the outer surface of the wall, the first pair of spaced apart first projections defining a first channel that extends to the rim, and the second pair of spaced apart second projections defining a second channel that extends to the rim;
a first spacer removably positioned in the first channel;
a second spacer removably positioned in the second channel; and
an extraction tool comprising a shaft, a first arm, and a second arm, the first arm and the second arm being attached to the shaft in a spaced apart relationship such that the first arm can engage the outer surface of the wall of the acetabular cup between the first pair of spaced apart first projections and the second arm can engage the outer surface of the wall of the acetabular cup between the second pair of spaced apart second projections.

32. The kit of claim 31 wherein:
the extraction tool further comprises an adjustable retention mechanism engaging a first section of the first arm and a second section of the second arm, the retention mechanism opposing opening forces exerted on the first arm and the second arm.

33. (canceled)

34. (canceled)

35. The kit of claim 31 wherein:
the extraction tool further comprises a handle slidable on the shaft and a stop on a proximal end of the shaft, the stop transmitting force from the handle to the shaft when the handle strikes the stop.

36. (canceled)

37. A kit for preparing a prosthetic system for providing motion between a pelvis and a femur of a hip joint of a subject, the kit comprising:
an acetabular cup including a wall terminating in a rim, the wall and the rim defining a cavity of the acetabular cup, the wall having an outer surface and an inner surface within the cavity of the acetabular cup, the acetabular cup having a first pair of spaced apart first projections extending outwardly from the outer surface of the wall and a second pair of spaced apart second projections extending outwardly from the outer surface of the wall, the first pair of spaced apart first projections defining a first channel that extends to the rim, and the second pair of spaced apart second projections defining a second channel that extends to the rim;
a first spacer removably positioned in the first channel;
a second spacer removably positioned in the second channel; and
an extraction tool comprising a frame, a first block moveable with respect to the frame, a second block moveable with respect to the frame, the first block and the second block being located in a spaced apart relationship in an interior space of the frame such that the first block can engage the outer surface of the wall of the acetabular cup between the first pair of spaced apart first projections and the second block can engage the outer surface of the wall of the acetabular cup between the second pair of spaced apart second projections.

38. The kit of claim 37 wherein:
the frame comprises a support, a first arm, and a second arm, the first arm and the second arm being attached to the support in a spaced apart relationship thereby defining the interior space of the frame.

39. (canceled)

40. (canceled)

41. (canceled)

42. (canceled)

43. The kit of claim 37 wherein:
the first block includes a first distal projection for engaging the outer surface of the wall of the acetabular cup; and
the second block includes a second distal projection for engaging the outer surface of the wall of the acetabular cup.

44. A kit for preparing a prosthetic system for providing motion between a pelvis and a femur of a hip joint of a subject, the kit comprising:
an acetabular cup including a wall terminating in a rim, the wall and the rim defining a cavity of the acetabular cup, the wall having an outer surface and an inner surface within the cavity of the acetabular cup;
a drill guide dimensioned such that a distal surface of the drill guide can engage the inner surface within the cavity of the acetabular cup, the drill guide having at least two passageways for directing a boring instrument into the wall of the acetabular cup for creating at least two throughholes in the wall of the acetabular cup; and
an extraction tool comprising:
(i) a frame,
(ii) a cap secured to the frame, the cap having a wall with at least two holes, the cap being dimensioned such that a distal surface of the cap can engage the inner surface within the cavity of the acetabular cup,
(iii) a pin in each hole of the cap, and
(iv) a shaft dimensioned to move each of the pins into an associated throughhole of the acetabular cup when the distal surface of the cap engages the inner surface within the cavity of the acetabular cup.

45. The kit of claim 44 further comprising:
a handle that engages the shaft for translating the shaft towards the cap and moving each of the pins into an associated throughhole of the acetabular cup.

46. The kit of claim 44 wherein:
the drill guide has a first passageway having a first longitudinal axis and a second passageway having a second longitudinal axis, and
the first longitudinal axis and the second longitudinal axis are in a reference plane.

47. The kit of claim 44 wherein:
the drill guide has a first passageway having a first longitudinal axis and a second passageway having a second longitudinal axis, and
the first longitudinal axis and the second longitudinal axis intersect.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)
